(19) United States
(12) Patent Application Publication
(10) Pub. No.: US 2026/0146020 A1
(43) Pub. Date: May 28, 2026

(54) PROCESSES FOR MAKING 1,2-DIACETOXYETHANE FROM MONOETHYLENE GLYCOL BY REACTIVE DISTILLATION

(71) Applicant: New Product Innovation, LLC, Johnston, IA (US)

(72) Inventor: Glenn Graham, Harrison, ID (US)

(73) Assignee: New Product Innovation, LLC, Johnston, IA (US)

(21) Appl. No.: 19/399,372

(22) Filed: Nov. 24, 2025

Related U.S. Application Data
(60) Provisional application No. 63/723,878, filed on Nov. 22, 2024.

(51) Int. Cl. C07C 67/08; B01D 3/00; B01D 3/14; C07C 67/54

(52) U.S. Cl. CPC C07C 67/08; B01D 3/009; B01D 3/143; C07C 67/54

(57) ABSTRACT

Disclosed herein are various embodiments pertaining to continuous processes for making ethylene glycol diacetate, also known as 1,2-diacetoxyethane, by esterification of monoethylene glycol to 2-hydroxyethyl acetate and the esterification of 2-hydroxyethyl acetate to ethylene glycol diacetate. Various embodiments herein use reactive distillation in processes promising enhanced economic viability in the production of high-quality ethylene glycol diacetate.

### CROSS-REFERENCES & RELATED APPLICATIONS

This application claims priority to U.S. Provisional Application No. 63/723,878 filed Nov. 22, 2024, and entitled “PROCESSES FOR MAKING 1,2-DIACETOXYETHANE FROM MONOETHYLENE GLYCOL BY REACTIVE DISTILLATION,” which is hereby incorporated by reference in its entirety under 35 U.S.C. § 119 (e).

### TECHNICAL FIELD

This disclosure pertains to processes for converting monoethylene glycol to 1,2-diacetoxyethane (ethylene glycol diacetate) by reactive distillation, and particularly to such reactive distillation processes having enhanced economics for production of high-quality ethylene glycol diacetate.

### BACKGROUND

Ethylene glycol diacetate has various uses itself, such as a solvent and ingredient in chemical formulations, and as an intermediate, e.g., for making vinyl acetate. Vinyl acetate monomer (VAM) is an important commodity chemical with an annual global production volume in 2020 of nearly 9 million metric tonnes. An interest exists in using sustainable feedstocks rather than fossil-based feedstocks to make commodity chemicals, including VAM. Commercial acceptance of commodity chemicals made using sustainable feedstocks will, in part, depend on the cost of their production.

Numerous processes exist for making VAM. Vinyl acetate is typically made by the catalytic reaction of ethylene, acetic acid, and oxygen. Some industrial production uses the hydroesterification of acetic acid and acetylene. Ethylene is typically made from fossil fuels although ethanol made from sustainable resources, can be converted to ethylene for use in the typical catalytic process. Another proposed route is the thermal or catalytic cracking of 1,1-diacetoxyethane. See, for instance, U.S. Pat. No. 2,425,389 and European Patent Application Publication No 0 048 173 A1. The high costs of producing 1,1-diacetoxyethane have not led to this proposed route achieving commercial viability for making VAM. U.S. Pat. No. 3,787,485 discloses the thermal cracking of ethylene glycol diacetate to vinyl acetate at 435° C. to 560° C. This process has also not seen commercial viability.

Nevertheless, the use of ethylene glycol diacetate to make VAM continues to be of interest as ethylene glycol diacetate can be made from monoethylene glycol which, in turn, can be made from sustainable resources. The conventional process for making monoethylene glycol is by cracking fossil-based feedstock to produce ethylene, partially oxidizing the ethylene to produce ethylene oxide and then hydrolyzing ethylene oxide to monoethylene glycol. The conventional process could use ethylene that is derived from sustainable feedstocks, e.g., ethylene made by the dehydration of ethanol derived from the fermentation of carbohydrates. Other processes exist for making monoethylene glycol from sustainable feedstocks. For instance, sustainable feedstock can be converted to syngas and monoethylene glycol can be made by the glycol oxalate process such as disclosed in U.S. Pat. No. 4,453,026. Also, recent efforts have been devoted to converting carbohydrates to monoethylene glycol by retro aldol conversion of the carbohydrate to, among others, glycol aldehyde, and hydrogenation to monoethylene glycol. See, for instance, U.S. Pat. No. 9,783,472.

In the esterification of monoethylene glycol with acetic acid, first the half ester, 2-hydroxyethyl acetate, is produced with water being the coproduct, and then the half ester is reacted with acetic acid to make ethylene glycol diacetate and water. Each of these reactions is an equilibrium reaction, and to drive the reactions toward completion, water is removed from the reaction menstruum.

Reactive distillation has long been proposed for esterification reactions as water is continuously removed from the reaction menstruum. Although reactive distillation holds promise for the conversion of monoethylene glycol to ethylene glycol diacetate, workers in the field have recognized that the equipment size and energy required for a reactive distillation unit to achieve an ethylene glycol diacetate product having an acceptably low concentration of 2-hydroxyethyl acetate and water, detract from the economic viability of reactive distillation for commercial scale operations.

Suman, et al., in Entrainer Based Reactive Distillation for Esterification of Ethylene Glycol with Acetic Acid, Ind. Eng. Chem. Res, 2009, 48, 9461-9470, discuss the use of an entrainer, ethylene dichloride, in a continuous reactive distillation column. In addition to facilitating the removal of water from the reactive distillation column, the ethylene dichloride entrainer is said to enable achieving complete separation of acetic acid from water. The mole fraction of ethylene dichloride in the reactive distillation column was about 0.5. The sizing of a reactive distillation column and heat duty for a commercial scale unit would have to take into account the large volume of the entrainer. Additionally, the entrainer serves to dilute the reactants and thus adversely affects the esterification reaction rates.

In 2016, Huang, et al., in Innovative Ethylene Glycol Diacetate Synthesis Process in a Single Reactive Distillation Column, Chemical Engineering and Processing, 109, 80-89, confirmed that it is not easy to obtain adequate purity of ethylene glycol diacetate by the esterification of monoethylene glycol with acetic acid. Huang, et al., disclose a proposed reactive distillation operation for making ethylene glycol diacetate from ethylene oxide and monoethylene glycol. Ethylene oxide is said to react with water to make ethylene glycol, and the authors state in section 5.1 that the esterification of ethylene glycol and the hydration of ethylene oxide “might promote each other”. Huang, et al., conclude, based upon computer simulations, that with ethylene oxide as the feed, high purity and yield of ethylene glycol diacetate can be reached with lower equipment costs and energy requirements. Ethylene oxide, however, is not a favored reactant for commercial operations unless proximate to a plant making ethylene oxide, and such plants typically use ethylene from fossil sources, not sustainable resources.

Accordingly, an interest remains to make ethylene glycol diacetate from monoethylene glycol, preferably derived from sustainable resources, on an economically feasible basis

### BRIEF SUMMARY

By this disclosure, processes for making ethylene glycol diacetate from monoethylene glycol and acetic acid by reactive distillation are provided that can provide enhanced economics. In accordance with this disclosure, it has been found that by feeding acetic acid at two or more certain locations into a reactive distillation zone, the reboiler duty required per unit of ethylene glycol diacetate is reduced as compared to substantially the same process but introducing all the acetic acid feed at a single location. Thus, those skilled in the art understanding the processes of this disclosure would understand that the advantages provided by the processes of this disclosure can be realized in practice in one or more ways including reduced energy costs, reduced capital costs and increase throughput volumes.

As a rule, a reactive distillation zone, i.e., the portion of the distillation column containing catalyst for esterification, can be characterized as having a plurality of distillation stages, each stage not only providing for the separation of liquid and vapor phases to separate components but also for the esterification reactions. The esterification reactions occurring in the reactive distillation zone also result in a change the mole ratios of the components, i.e., acetic acid, monoethylene glycol, 2-hydroxyethyl acetate and ethylene glycol diacetate. Hence, the upper section of the reactive distillation zone has a higher molar concentration of water and monoethylene glycol than a lower section of the reactive distillation zone. The lower section has a higher concentration of ethylene glycol diacetate and a lower concentration of water and monoethylene glycol than does the upper section. An intermediate section between the upper section and the lower section, typically has a higher content of 2-hydroxyethyl acetate than do the upper section and lower section. The number of distillation stages in the lower zone will depend upon a number of factors including, but not limited to, the sought concentration of 2-hydroxyethyl acetate in the tails and design choices as more distillation stages can be added than are the minimum required to achieve the conversion of monoethylene glycol to ethylene glycol diacetate. In general, at least 7 distillation stages are contained in the lower section. Frequently, the lower section contains distillation stages that have a mass ratio of ethylene glycol diacetate to 2-hydroxyethyl acetate of at least about 10:1 in the liquid phase. The upper section contains distillation stages that have a mass ratio of ethylene glycol diacetate to 2-hydroxyethyl acetate of less than about 0.05:1 in the liquid phase. Within the intermediate section is a transition section where the distillation stages have a mass ratio of ethylene glycol diacetate to 2-hydroxyethyl acetate of between about 0.1:1 and 5:1, optionally between about 0.1:1 and 1.5:1, in the liquid phase.

Without wishing to be bound by theory, a number of design and operational factors are believed to affect the performance of the reactive distillation zone and achieve the sought conversion of monoethylene glycol to ethylene glycol diacetate. These factors include, but are not limited to, the number of distillation stages, the liquid phase hold-up in the stages, catalytic activity, the amount of acetic acid that is allowed to pass as overhead from the distillation column, the reboiler duty, the amount of excess acetic acid, temperature and pressure. The operator typically looks at temperature, pressure, the distillate to feed ratio, and reflux ratio as the primary variables to optimize the operation of the reactive distillation zone. The processes of this disclosure provide another dimension to enhancing the performance of a reactive distillation zone for making ethylene glycol diacetate from monoethylene glycol.

In a broad aspect, various embodiments in this disclosure pertain to continuous processes for making ethylene glycol diacetate by esterification of monoethylene glycol to 2-hydroxyethyl acetate and the esterification of 2-hydroxyethyl acetate to ethylene glycol diacetate, comprising continuously feeding monoethylene glycol and acetic acid to a reactive distillation zone having a plurality of distillation stages, said reactive distillation zone having an upper section, an intermediate section and a lower section and being under reactive distillation conditions including the presence of esterification catalyst to provide an overhead from the upper section of the reactive distillation column comprising water and acetic acid, which overhead is continuously withdrawn from the reactive distillation zone, and a bottoms fraction containing ethylene glycol diacetate, which bottoms fraction is continuously withdrawn from the lower section, wherein at least about 90 percent of the monoethylene glycol is converted to ethylene glycol diacetate, and wherein the monoethylene glycol is fed to the upper section of the reactive distillation zone and a portion, in some embodiments at least about 40 percent, of the acetic acid is fed to the lower section of the reactive distillation zone and a portion, in some embodiments at least about 20 percent, of the acetic acid is fed to the intermediate section, optionally then to the transition section, of the reactive distillation zone.

By introducing the acetic acid at two or more locations in the reactive distillation zone, at least one location being in the lower section of the reactive distillation zone and at least one location being in the intermediate section of the reactive distillation zone, with all else being substantially the same, a reduction in boiler duty is achieved as compared to introducing the acetic acid at one location into the reactive distillation zone. In advantageous aspects of this disclosure, a portion of the acetic acid is introduced into the transition section of the intermediate section. In some embodiments, at least about 20 percent of the acetic acid feed is introduced into the transition section of the reactive distillation zone. In further embodiments, between about 20 and 40 percent of the acetic acid feed is introduced into the transition section of the reactive distillation zone.

The upper section of the reactive distillation zone contains a plurality of distillation stages having a top most stage, that is the first stage where monoethylene glycol, acetic acid and esterification catalyst are present. At least a portion of the monoethylene glycol, such as but not limited to at least about 75 percent, may be fed to the top-most distillation stage of the reactive distillation zone. The monoethylene glycol can be directly fed to the reactive distillation stage or indirectly fed, e.g., by being fed to a non-reactive rectification zone that receives the overhead from the reactive distillation zone and then passes downwardly as a liquid phase to the reactive distillation zone. The lower section of the reactive distillation zone also contains a plurality of distillation stages with the last distillation stage containing ethylene glycol diacetate, acetic acid, 2-hydroxyethyl acetate and esterification catalyst in the liquid phase being the bottom-most stage. The top of the lower section is the upper most distillation stage having in the liquid phase, a mass ratio of ethylene glycol diacetate to 2-hydroxyethyl acetate greater than about 10:1. In most instances, the lower section comprises at least 3 distillation stages, and acetic acid fed to at least one of the bottom-most distillation stages, often the bottom-most distillation stage.

In another broad aspect of this disclosure, continuous processes are disclosed for making ethylene glycol diacetate from monoethylene glycol and acetic acid by reactive distillation comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and acetic acid and to provide an ethylene glycol diacetate product;
b. continuously withdrawing the overhead containing water from the reactive distillation zone; and
c. continuously withdrawing from the reactive distillation zone ethylene glycol diacetate product, wherein the overhead from the reactive distillation zone contains acetic acid and is subjected to fractional distillation in a non-reactive rectification zone having a substantial absence of esterification catalyst, to separate water as an overhead and acetic acid as a liquid phase passing to the reactive distillation zone, and between about 10 to 50 percent of the monoethylene glycol supplied to the reactive distillation zone is passed to the non-reactive distillation zone and becomes a portion of the liquid phase passing to the reactive distillation zone.

Without wishing to be bound by theory, it is believed that the monoethylene glycol, which will primarily be in the liquid phase under the conditions of the non-reactive distillation zone, assists in the separation of acetic acid from water.

At least one specification heading is required. Please delete this heading section if it is not applicable to your application. For more information regarding the headings of the specification, please see MPEP 608.01(a).

### DETAILED DESCRIPTION

All patents, published patent applications and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3. 0.63, 0.815 and so forth) are included as are all intermediate ranges (e.g., 0.2-0.5, 0.54-0.913, and so forth).

The use of the terms “a” and “an” is intended to include one or more of the elements described.

Admixing or admixed means the formation of a physical combination of two or more elements which may have a uniform or non-uniform composition throughout and includes, but is not limited to, solid mixtures, solutions and suspensions.

Acetic acid providing feed is a feed containing acetic acid and/or precursors to acetic acid that will form acetic acid under esterification conditions such as acetic anhydride.

The term “Available Hydroxyls” means the hydroxyls on C2 to C4 hydrocarbons such as monoethylene glycol, 2-hydroxyethyl acetate, 1,2-propanediol, 1,2-butanediol, and the like.

A crude ethylene glycol diacetate product is an ethylene glycol diacetate-containing mixture at a point or region in the process and may or may not be recovered or isolated as a discrete product. Thus, it can be a point or region in a flowing stream such as the liquid phase in a distillation column, in the bottoms section of a distillation column or a flowing liquid phase in a reboiler.

A distillation plate or stage is a theoretical distillation plate ascertained by calculation. Where no reaction is occurring, the theoretical plate is where physical thermodynamic equilibrium is achieved between the vapor and liquid phases. Where a reaction is occurring, as in the reactive distillation zone, physical equilibrium is achieved but not chemical equilibrium as the rate of esterification slows approaching equilibrium. Often, at steady-state the composition of the liquid phase is at about 40 to 90 percent toward the equilibrium composition. For purposes of the reactive distillation zone, the physical thermodynamic equilibrium is for water and acetic acid in the vapor phase and in the liquid phase, and the chemical equilibria are for water, acetic acid, monoethylene glycol, 2-hydroxyethyl acetate and ethylene glycol diacetate in the liquid phase.

Ethylene glycol diacetate is also known as 1,2-diacetoxyethane.

The half acetate ester of ethylene glycol is also known as 2-hydroxyethyl acetate.

Hold-up is the quantity, or volume, of liquid phase that can be introduced into the reactive distillation zone without leaving the reactive distillation zone. The hold-up is influenced by the configuration of the internal structure of the zone. For instance, bubble trays will have a greater hold-up quantity than structured packing.

Monoethylene glycol is sometimes referred to herein as ethylene glycol.

Reactive distillation is a process in which reaction and separation occur simultaneously on some or all of the stages of a distillation column, and reaction products (e.g., ester and water) are separated and removed. The continuous removal of the reaction products via the distillation can increase conversion beyond that of the equilibrium composition in a batch reactor. Thus, as feedstock (monoethylene glycol) and esterification catalyst, which are introduced on an upper distillation stage of the reactive distillation zone, contact acetic acid rising in the vapor phase from a lower distillation stage where it has been introduced, the esterification reaction occurs, producing one or more acetate esters and water. The acetate esters, having a relatively high boiling point, remain mostly in the flowing downward-flowing liquid phase, whereas water, having a relatively low boiling point, mostly passes into the upward-flowing vapor phase. As the liquid progresses downward to lower distillation stages of the reaction zone, 2-hydroxyethyl acetate and unreacted monoethylene glycol continue to react with the acetic acid rising from lower distillation stages, forming additional esters and water. The water, rising in the vapor from lower distillate stages, combines with water generated in the upper distillation stages. There being a vapor-liquid equilibrium relationship for all of the components, the water is present in the liquid phase in varying concentrations but tends to be present in lower concentrations in the liquid for lower distillation stages than it is in the upper distillation stages. Conversely, the concentration of acetic acid in both phases gradually decreases as it progresses upward in the column and is reacted away by the esterification reaction. The bottom (last reaction zone) of the distillation column can, therefore, have a liquid phase rich in ethylene glycol diacetate and very lean in water and 2-hydroxyethyl acetate, whereas the water leaving the top of the column (non-reactive rectification section) as distillate tends to have a low concentration of acetic acid.

Substantially non-reactive means that under the reaction conditions including time, temperature and presence of catalyst and other adjuvants, less than 1 percent of the moiety would be reacted.

Sustainable resources are plants and animals, including, but not limited to, waste products from plants and animals, and sustainable feedstocks are derived from sustainable resources. Sustainable resources also include carbon dioxide used as a feedstock in processes to make monoethylene glycol, whether that carbon dioxide is captured from direct air capture or from emissions from facilities that emit carbon dioxide, including, but not limited to, incineration, power generation, fermentations, and chemical and other industrial processes.

Vicinal glycols are 1,2-dihydroxyalkanes and lower vicinal glycols are monoethylene glycol, propylene glycol and 1,2-butanediol.

### Feedstocks

With respect to the esterification to produce ethylene glycol diacetate, in its broad aspects, the disclosed process contemplates the monoethylene glycol feedstock being generated from any process using any suitable raw material. Advantageously at least a portion of one or both of monoethylene glycol and acetic acid used to make ethylene glycol diacetate are derived from sustainable resources, and optionally at least about 75 percent is derived from sustainable resources. The monoethylene glycol-containing feedstock can be derived from any suitable source. For instance, ethylene can be obtained from sustainable feedstocks and then converted to monoethylene glycol according to the conventional process of partial oxidation to ethylene oxide and hydrolysis to monoethylene glycol. The conventional process provides monoethylene glycol substantially free of propylene glycol and 1,2-butanediol. Alternative processes for making monoethylene glycol from sustainable feedstocks can have greater efficiencies of carbon in the biomass to monoethylene glycol but have challenges in securing high purities. For instance, a sustainable feedstock can be converted to syngas and monoethylene glycol can be made by the glycol oxalate process such as disclosed in U.S. Pat. No. 4,453,026. The glycol oxalate process also generates impurities including 1,2-butanediol. Carbohydrates can be converted to monoethylene glycol by retro aldol conversion of the carbohydrate to, among others, glycol aldehyde, and hydrogenation to monoethylene glycol (Retro Aldol Process). See, for instance, U.S. Pat. No. 9,783,472. This process can have a high selectivity to monoethylene glycol, but produces, among others, vicinal glycols including, but not limited to, propylene glycol and 1,2-butanediol. The separation of monoethylene glycol from other vicinal glycols can be energy intensive, especially 1,2-butanediol which has substantially the same normal boiling point as monoethylene glycol. Certain processes herein integrate with the production of monoethylene glycol from certain processes, such as the Retro Aldol Process and the glycol oxalate process, for purposes of removing propylene glycol and 1,2-butanediol by removing their reaction products.

The acetic acid, which may be derived from sustainable resources, such as produced by the fermentation of sugars, or fossil feedstocks, can be supplied to process in any suitable form including glacial acetic acid or aqueous solutions of acetic acid. The acetic acid feed may also contain acetic anhydride, but usually only in a minor amount, and most often if present, up to 10 mass percent, of the acetic acid feed to the reactive distillation is acetic anhydride. For purposes herein, the amount of acetic acid is based upon the theoretical moles of acetic acid that can be derived from the form of acetic acid supplied.

### Reactive Distillation Process Conditions

The acetic acid is provided in at least a stoichiometric amount required to form the diacetate of the monoethylene glycol and diacetates of any other lower vicinal glycols in the feedstock. Usually, the total amount of acetic acid including acetic anhydride provided per unit time to the reaction system (excluding any acetic acid steady state inventory, that is, acetic acid which is retained in the reactive distillation zone in the hold-up) is less than about 1.5, say, about 1 to 1.15, times that required on a stoichiometric basis to convert the Available Hydroxyls in the monoethylene glycol containing feedstock provided per that unit time to the corresponding acetate esters. The excess acetic acid will pass to the bottom of the reactive distillation zone and can, if desired, be recovered from the bottoms of the reactive distillation zone and recycled. Some, usually a very minor amount, of acetic acid can be contained in the water fraction from the overhead, and some can be contained in purge streams.

In various implementations, the reactive distillation zone contains at steady state operation, an inventory of acetic acid (“steady state inventory acetic acid”) which is in addition to the amount of acetic acid fed. Where the steady state inventory acetic acid results in a higher or lower mole ratio of acetic acid to Available Hydroxyls in the liquid phase the driving force for the esterifications is increased or decreased respectively. Regardless, an increase in the liquid hold-up due to an increase in the amount of the steady state inventory acetic acid at any given distillation stage serves to increase the liquid residence time in that stage giving more time for the esterifications to proceed toward chemical equilibria. The amount of the steady state inventory acetic acid depends upon the configuration and operation of the reactive distillation as does the distribution of the steady state inventory acetic acid. Often, the steady state inventory acetic acid in the vapor phase and in the liquid phase in the reactive distillation zone as calculated as moles of acetic acid to moles of Available Hydroxyls in the feedstock is greater than 0.1:1 or 0.2:1, say, about 0.25:1 to 2:1. It is to be understood that the concentration of acetic acid in any given distillation stage will be determined by the distillation operating parameters and can vary widely over the height of the reactive distillation zone. For example, this ratio may be as low as 0.0001:1 in the liquid phase at the top most distillation stage and as high as 100:1 in the liquid phase at the bottom most stage of the reactive distillation zone.

This steady state inventory acetic acid can be maintained in the reaction system by any suitable means, e.g., by using one or more of the following: optimizing the acetic acid feed location, increasing the boil-up rate in the column, retaining acetic acid in the reaction system through packing or tray design and recycling excess acetic acid recovered from the reaction system. For example, the use of, for example, bubble trays and valve trays, which can have large liquid hold-ups, in the reactive distillation zone can assist in maintaining the steady state inventory acetic acid.

In general, the greater the hold-up in a distillation stage, the closer the liquid phase comes to equilibrium for the liquid composition at that stage. However, increased liquid hold-up usually comes with a penalty of increased pressure drop. Increasing catalytic activity also results in pushing the esterification reactions closer to equilibrium for the liquid composition at that stage, but costs associated with the use of more catalyst have to be taken into account. Hence, with higher catalytic activity, the hold-up can be reduced while obtaining substantially the same performance as with a higher hold-up. It is also to be understood that the design of the reactive distillation zone can have different sections with different hold-ups per distillation stage. For instance, distillation stages in the upper and the lower sections of the reactive distillation column generally have lower rates toward equilibrium than those in the intermediate section. Accordingly, the distillation stages in at least one of the upper section and the lower section of the reactive distillation zone could be designed to have a higher hold-up with the intermediate section having a lower hold-up, and thereby attenuate pressure drop but yet still obtain the same conversion to ethylene glycol diacetate. Frequently, the hold-up averages up to about 1, say, about 0.005 to 0.5, and more often about 0.01 to 0.4, liter of liquid per kilogram per hour of monoethylene glycol in the feed per distillation stage.

The monoethylene glycol feedstock is subjected to acidic esterification conditions in the presence of acetic acid to provide ethylene glycol diacetate. Acidic esterification conditions include the presence of an acidic catalyst and elevated temperatures. Suitable esterification catalysts have a pKa of less than about 3, often less than about-1, and are not unduly reactive with monoethylene glycol. The esterification catalysts may be heterogeneous or homogeneous, and examples of catalysts include sulfonic membranes such as Nafion™ sulfonated tetrafluoroethylene-based fluoropolymer, solid Brønsted acids such as graphene supports having functional groups (one or more of SO3H—, COOH—, and phenolic OH—) thereon, methane sulfonic acid, p-toluene sulfonic acid, and trifluoromethanesulfonic acid. Esterification catalysts also include inorganic acids such as phosphoric acid, phosphonic acid, sulfuric acid sulfonic acid, sulfoacetic acid and mixtures thereof. The catalyst is employed in a catalytically effective amount, generally for homogeneous catalysts, in the range of about 0.01 to 10 grams of catalyst per 100 grams of liquid phase in the reactive distillation zone. Heterogeneous catalysts are often provided such that the mass hourly space velocity of monoethylene glycol feed to mass of heterogeneous catalyst is in the range of about 0.01 to 50, say about 0.05 to 20, hour-1. The heterogeneous catalyst can be distributed uniformly per each distillation stage in the reactive distillation zone, or non-uniformly such that at least one distillation stage contains a greater amount of heterogeneous catalyst than does at least one other distillation stage.

The esterification is conducted in the liquid phase within the reactive distillation zone. Vapor and liquid phases must be present on all distillation stages of the reactive distillation zone for the separations to occur, so, for a given column pressure, the temperature on each distillation stage will be the boiling point of the liquid mixture on that stage and is dependent upon the composition of that mixture. If an azeotroping agent (entrainer) is used, then the boiling points of the distillation stages where the azeotroping agent is present will be affected by the presence of the azeotroping agent. Thus, water, which is a coproduct of the esterification is removed as a vapor in the reactive distillation zone thereby driving the esterification reactions towards completion.

In some instances, a component that azeotropes with water is present and serves to reduce the temperature required to remove water. Examples of azeotroping agents for the dehydration of acetic acid are known, see, for instance, U.S. Pat. No. 5,160,412, and for the reactive distillation of ethylene glycol with acetic acid, are known, see, for instance Suman, et al., Entrainer Based Reactive Distillation for Esterification of Ethylene Glycol with Acetic Acid, Ind. Eng. Chem. Res., 2009, 48, 9461-9470. As will be discussed later, the use of azeotroping agents in the reactive distillation dilute the reactants and can increase capital and heat duty. In some embodiments of this disclosure, an azeotroping agent, when used, is one that has a normal boiling point at least about 25° C. below the normal boiling point of acetic acid and thus the azeotroping agent is substantially not present in the reactive distillation column below the point of introduction of the azeotroping agent.

The esterification conditions, in various implementations, include a pressure in the range of about 10 to about 500, optionally 50 to 250, kilopascals absolute, and maximum temperatures in the reactive distillation (excluding the still bottoms and any reboiler that provide heat to the distillation column) are in the range of about 80° C. to about 250° C. In further implementations, the maximum reactive distillation temperatures can be in the range of about 90° C. to about 200° C.

### Reactive Distillation Operation

It is to be understood that the reactive distillation zone may reside in a single column, which may be beneficial, or two or more distillation columns, when the process design requires. The monoethylene glycol feedstock is typically provided to the top-most distillation stage or stages in the reactive distillation zone. A portion of the distillation column above the reactive distillation zone (non-reactive rectification section) is for primarily for the removal of acetic acid from water but also serves to remove monoethylene glycol and esters that pass to the non-reactive rectification section. If desired, a portion of monoethylene glycol of the feedstock, e.g., about 10 to 50 mass percent of the total monoethylene glycol feedstock, can be fed to the non-reactive rectification section below its top distillation stage. As the normal boiling point of monoethylene glycol is about 97° C. higher than that of water, it passes downwardly in the non-reactive rectification section as a liquid and back to the reactive distillation zone. This monoethylene glycol is believed to assist in pushing the acetic acid down in the distillation column, and thus serves to reduce capital and heat duty for this portion of the distillation column.

In accordance with this disclosure, the acetic acid feedstock is introduced at any location or at two or more locations in the reactive distillation zone. Surprisingly, the use of two or more locations of acetic acid feed can provide a lower heat duty than if the feed were only provided to a single distillation stage. The use of two or more locations of acetic acid feed can be implemented to alter the acetic acid concentrations in the liquid phase as compared to a single introduction location, yet still achieve desirably low amounts of acetic acid passing into the overhead.

The acetic acid feed to the reactive distillation zone is that fresh acetic acid supplied as make-up for the acetic acid reacted, and lost in the overhead, ethylene glycol diacetate product stream, purge streams, and otherwise, and that acetic acid recovered and recycled to the reactive distillation zone. Where acetic acid is recovered and recycled, it may be included in the feed to one or more of the locations in the reactive distillation zone. The acetic acid fed to the reactive distillation zone can be in the vapor or liquid or mixed phases. Typically, the feed is substantially in the liquid phase immediately prior to being introduced into the reactive distillation zone although a portion of the feed may be vaporized upon entry into the reactive distillation zone. Advantageously, the acetic acid feed is at a temperature greater than 20° C. below that of, and most often the feed is within 5° C. of the distillation stage into which it is being introduced. The introduction of acetic acid into a distillation stage will increase the concentration of acetic acid in the distillation stage and enhance the driving force to esterification, and the perturbation will affect not only the distillation stage into which the acetic acid is introduced, but also distillation stages above and below that distillation stage.

A portion of the acetic acid feed is provided to the lower section of the reactive distillation zone and a portion of the acetic acid feed is provided to the intermediate section, optionally the transition section, of the reactive distillation zone. In various embodiments, at least about 40 percent of the acetic acid feed is provided to the lower section, and in the lower section, that portion of the acetic acid feed can be introduced into one or more distillation stages. In further embodiments, between about 50 and 90 percent of the acetic acid feed is provided to the lower section. In still further embodiments, between about 50 and 80 percent of the acetic acid feed is provided to the lower section.

This portion of the acetic acid feed may be introduced into one or more of the bottom-most three distillation stages. In some embodiments, at least a portion of this portion of the acetic acid feed, may be introduced into the bottom-most distillation stage and/or next to bottom-most distillation stage. In further embodiments, at least about 50 to about 100 percent of the portion of the acetic acid feed, may be introduced into the bottom-most distillation stage and/or next to bottom-most distillation stage.

Surprisingly, even though a very significant portion of the acetic acid feed is introduced into the lower section of the reactive distillation zone, the heat duty is still able to be reduced as compared to substantially the same process but where all the acetic acid feed is introduced at a single point higher in the reactive distillation zone.

In various embodiments, a portion of the acetic acid feed is provided at one or more locations in the intermediate section of the reactive distillation zone. In some embodiments, the portion of acetic acid fed is at least about 10 percent. In further embodiments, the portion of acetic acid fed is at least about 15 percent. In still further embodiments, the portion of acetic acid fed is about 20 to 60 percent.

In some embodiments, the acetic acid fed to the intermediate zone may be introduced into a distillation stage having a mole ratio in the liquid phase of ethylene glycol diacetate to 2-hydroxyethyl acetate between about 0.1:1 and 5:1. In further embodiments, the mole ratio in the liquid phase of ethylene glycol diacetate to 2-hydroxyethyl acetate may be between about 0.1:1 to 1.5:1. In still further embodiments, the mole ratio in the liquid phase of ethylene glycol diacetate to 2-hydroxyethyl acetate may be between about 0.15:1 to 1:1. Where two or more distillation stages in the intermediate section receive acetic acid feed, the lower feed location may be a distillation stage having a mole ratio in the liquid phase of ethylene glycol diacetate to 2-hydroxyethyl acetate between about 0.1:1 and 5:1, and the upper feed location may be a distillation stage having a mole ratio in the liquid phase of ethylene glycol diacetate to 2-hydroxyethyl acetate between about 0.1:1 and 1:1. In some embodiments, the distillation stages receiving the acetic acid feed are at least 3 distillation stages apart, and for operations using two acetic acid feed points, optionally at least 5 distillation stages apart. Where two or more acetic acid feed locations are used, the relative portions of the feeds will depend upon the locations of introduction and the split between the feed to the lower section and the intermediate section of the reactive distillation zone. In some embodiments, the upper most feed location of the multiple feeds into the intermediate section provides up to 30 percent of the total acetic acid feed, which may be in the transition section. In further embodiments, the upper most feed location of the multiple feeds into the intermediate section provides between about 5 and 25 percent of the total acetic acid feed, which may be in the transition section. In such embodiments, the lower most feed location in the intermediate section may receive up to about 30 percent of the total acetic acid feed. In further embodiments, the lower most feed location in the intermediate section may receive about 5 to 25, percent of the total acetic acid feed.

In various embodiments, where the lowermost feed location in the intermediate section is below the transition section, some advantage is received; however, a greater benefit may be obtained by introducing that portion of the acetic acid feed into the transition section or lower section.

The advantages of the processes of this disclosure are additive to optimizations of reactive distillation columns as are known in the art. Variables considered in the prior art include the fraction of acetic acid in the overhead, the concentration of 2-hydroxyethyl acetate in the bottoms fraction, temperature, pressure, reflux ratio, number of distillation stages, use of entrainers, type of packing or trays, hold-up, steady state acetic acid inventory acetic acid, acetic acid feed rate, and catalytic activity (type of catalyst and amount). By way of example and not in limitation, the greater the hold-up in a distillation stage, the closer the liquid phase comes to equilibrium for the liquid composition at that stage. However, increased liquid hold-up usually comes with a penalty of increased pressure drop. Increasing catalytic activity also results in pushing the esterification reactions closer to equilibrium for the liquid composition at that stage, but costs associated with the use of more catalyst have to be taken into account. Hence, with higher catalytic activity, the hold-up can be reduced while obtaining substantially the same performance as with a higher hold-up. It is also to be understood that the design of the reactive distillation zone can have different sections with different hold-ups per distillation stage. For instance, distillation stages in the upper and the lower sections of the reactive distillation column generally have lower rates toward equilibrium than those in the intermediate section. Accordingly, the distillation stages in at least one of the upper section and the lower section of the reactive distillation zone could be designed to have a higher hold-up with the intermediate section having a lower hold-up, and thereby attenuate pressure drop but yet still obtain the same conversion to ethylene glycol diacetate.

The separation of water from acetic acid is accomplished by fractional distillation in the non-reactive rectification section of the column. The number of distillation stages used will, in part, be determinative of the concentration of acetic acid in the water overhead. Since acetic acid is readily susceptible to degradation in wastewater treatment facilities, the primary consideration of the operator is the cost of the acetic acid and the energy and capital costs for the water and acetic acid separation. In general, the concentration of acetic acid in the water overhead is desirably less than about 1 mass percent.

The use of azeotropic distillation has been proposed to achieve enhanced separations. See, for instance, Suman, et al., discussed above. In various aspects of the processes of this disclosure, certain azeotroping agents, or entrainers, are present in the non-reactive rectification section of the reactive distillation column. In some embodiments, entrainers may be characterized as having a normal boiling point of less than about 115° C., a normal azeotropic minimum boiling point no greater than about 80° C., a mutual solubility of water in the entrainer (at 25° C.) of less than about 5 mole percent, a mutual solubility of the entrainer in water of less than about 5 mole percent, and a substantial lack of reactivity in the reaction menstruum in the reactive distillation column. In further embodiments, the entrainers may be characterized by a mutual solubility in water of less than about 2 mole percent.

Examples of entrainers include, but are not limited to, heptane, ethylene dichloride, cyclohexane, benzene, di-isopropyl ether, and hexane. In various embodiments, the entrainer is introduced into the reactive distillation column at or above the distillation stage to which monoethylene glycol feedstock is introduced where it rapidly becomes vaporized either as itself or as an azeotrope. Thus, the entrainer can be introduced at a distillation stage in the reactive distillation zone to which monoethylene glycol is added or into a distillation stage in the non-reactive rectification zone. Where monoethylene glycol is introduced into the non-reactive rectification sone, the entrainer can be introduced into the non-reactive entrainer zone at the same distillation stage, or at a distillation stage below or above that distillation stage, as the monoethylene glycol is introduced. Thus, the entrainer does not unduly dilute the reactants in the reactive distillation portion of the distillation column nor require undue increases in heat duty. The water and azeotrope in the overhead can be condensed, and via liquid phase separation, an aqueous phase can be directed to wastewater treatment and the organic liquid phase can be recycled to the reactive distillation column. In an exemplary operation, the entrainer has little solubility in water and is present in an amount sufficient to provide at the upper distillation stage or stages of the non-reactive rectification zone, two liquid phases which can be refluxed back to the non-reactive rectification zone. Decanting of the two liquid phases can be undertaken with recycling the organic liquid phase to the non-reactive rectification zone.

In various embodiments, the reactive distillation zone may provide as a product, a liquid phase having acetic acid and ethylene glycol diacetate as the predominant components, at least 90 mass percent of the liquid phase. In further embodiments, the reactive distillation zone may provide as a product, a liquid phase having acetic acid and ethylene glycol diacetate as the predominant components between about 95 to 99.9 mass percent of the liquid phase. This liquid phase may also contain 2-hydroxyethyl acetate and water, and it can contain esterification catalyst, and would contain esterification catalyst if a homogeneous catalyst is used.

The operator has the flexibility to adjust the concentration of 2-hydroxyethyl acetate in the esterification product primarily by one or more of (i) setting the number of distillation stages in the reactive distillation zone, with fewer resulting in increased 2-hydroxyethyl acetate concentration, (ii) setting the reflux ratio, with lower reflux ratios resulting in increased 2-hydroxylethyl acetate concentration, (iii) setting the hold-up in the reactive distillation zone, with lower hold-up, i.e., reduced steady state inventory acetic acid, resulting in increased 2-hydroxyethyl acetate concentration. In general, the conditions providing a higher concentration of 2-hydroxyethyl acetate in the esterification product beneficially provide one or more of capital cost and heat duty cost savings. The concentration of 2-hydroxyethyl acetate in the esterification product may be at least about 0.1 or 0.2 mass parts per 100 mass parts of ethylene glycol diacetate. In further embodiments, the concentration of 2-hydroxyethyl acetate in the esterification product may be between about 0.2 or 0.5 and 5 mass parts per 100 mass parts of ethylene glycol diacetate. In still further embodiments, the concentration of 2-hydroxyethyl acetate in the esterification product may be between about 0.5 and 4 mass parts per 100 mass parts of ethylene glycol diacetate. The concentration of water in the esterification product may be less than about 0.5 mass parts per 100 mass parts of ethylene glycol diacetate. In further embodiments, the concentration of water in the esterification product may be less than about 0.1 or 0.2 mass parts per 100 mass parts of ethylene glycol diacetate.

The liquid phase from the fractional distillation section of the reactive distillation zone is typically passed to a bottoms section or kettle. The bottoms section can be heated to provide a heated vapor stream to pass into the reactive distillation zone, provide the heat energy for the distillation, and provide a residual liquor containing ethylene glycol diacetate. Alternatively, the liquid phase can be passed to a reboiler to provide the heated vapor phase to be returned to the reactive distillation zone. In either event, a significant portion of the acetic acid may be returned to the reactive distillation zone.

As discussed in copending United States provisional patent application with attorney docket number 9022034-194046, filed on even date herewith, the esterification can be operated such that appreciable 2-hydroxyethyl acetate still remains in the esterification product and acetic anhydride added to react the 2-hydroxyethyl acetate to ethylene glycol diacetate. The acetic anhydride can also react with water and catalyst present. This esterification product is also referred to herein as the crude ethylene glycol diacetate product.

Acetic anhydride may be contacted with a crude ethylene glycol diacetate product to provide a treated product having a lower concentration of 2-hydroxyethyl acetate and a lower concentration of water. Where used, the molar ratio of acetic anhydride to the total moles of 2-hydroxyethyl acetate and water, may be sufficient to reduce the concentration of 2-hydroxyethyl acetate and water in the treated ethylene glycol diacetate product to desired levels. In some embodiments, the desired levels of ethylene glycol diacetate may be less than 0.1 mass parts of 2-hydroxyethyl acetate per 100 mass parts of ethylene glycol diacetate and less than 0.05 mass parts of water per 100 mass parts of ethylene glycol diacetate. In further embodiments, the desired levels of ethylene glycol diacetate may be less than 0.05 mass parts of 2-hydroxyethyl acetate per 100 mass parts of ethylene glycol diacetate less than 0.01 mass parts of water per 100 mass parts of ethylene glycol diacetate.

In various embodiments, the amount of acetic anhydride provided is usually sufficient to reduce the amount of 2-hydroxyethyl acetate by at least about 50 percent. In further embodiments, the amount of acetic anhydride provided is usually sufficient to reduce the amount of 2-hydroxyethyl acetate by at least about 75, percent. In various embodiments, the amount of acetic anhydride introduced may be in a mole ratio to the total moles of 2-hydroxyethyl acetate, water and acid groups (if any from a homogeneous acid catalyst) in the crude ethylene glycol diacetate product of at least about 0.4:1. In further embodiments, the amount of acetic anhydride introduced may be in a mole ratio to the total moles of 2-hydroxyethyl acetate, water and acid groups in the crude ethylene glycol diacetate product of at least at least about 0.5:1. In still further embodiments, the amount of acetic anhydride introduced may be in a mole ratio to the total moles of 2-hydroxyethyl acetate, water and acid groups in the crude ethylene glycol diacetate product of between about 1:1 to 2.5:1.

The reaction of acetic anhydride with 2-hydroxyethyl acetate proceeds quickly, especially at the typical temperature of and presence pf acid catalyst in, the distillation stage into which the acetic anhydride is introduced. Unreacted acetic anhydride, which often is that amount in excess of the stoichiometric amount to react with 2-hydroxyethyl acetate, water and acid catalyst, can be recycled for use in the process. Acetic anhydride has a normal boiling point of about 140° C., which is lower than that of ethylene glycol diacetate (about 186° C.), and thus, if desired, can be separated as a vapor and passed to the reactive distillation zone as a source of acetic acid or as a portion of the acetic anhydride used to contact the crude ethylene glycol diacetate product.

### EXAMPLES

A series of reactive distillation zone operations are modeled using Aspen Plus Version 14 from Aspen Technology. The following parameters are used for the modeling:
Distillation stages: except as otherwise stated, 30 (4 stages in a non-reactive rectification zone, which includes the condenser; 25 stages above the bottoms section; and one stage being the bottoms section which is a reactive distillation stage)
Monoethylene glycol: Fed to distillation stage 5.
Catalyst: Sulfuric acid introduced into distillation stage 5 to provide a concentration of 0.2 percent by mass based upon the mass of the liquid phase in the reactive distillation zone.
   Liquid hold-up per distillation stage: 0.2 liters per kilogram per hour of monoethylene glycol in the feed.
   Pressure: 101 kilopascal at the top of the non-reactive rectification zone.
   Temperature: 100° C. at the top of the non-reactive rectification zone.
   Monoethylene glycol feed rate: 26,350 kilograms per hour.
   Acetic acid feed rate: 53900 kilograms per hour.
   Acetic acid in overhead: 15 kilograms per hour.
   Acetic acid in liquid phase at distillation stage 29:0.286 kilogram per kilogram of ethylene glycol diacetate
   2-hydroxyethyl acetate in liquid phase at distillation stage 29:1.64 mass percent based on total 2-hydroxyethyl acetate and ethylene glycol diacetate.
   Monoethylene glycol conversion: Essentially completely reacted.

The Aspen simulation kinetics are confirmed by the operation of a laboratory-scale, glass Oldershaw column having an inside diameter of 28 millimeters and approximately 25 distillation stages including the condenser and reboiler and a single point of acetic acid feed. Due to the low hold-up, the catalyst concentration is increased to 4 mass percent of the liquid in the lower portion of the column.

The results of the simulation using one or two feed points are provided in Table I. In the Tables GDA is ethylene glycol diacetate and HEA is 1-hydroxyethyl acetate.

TABLE 1

| | Distillation | | Distillation | | Fraction | Reboiler |
|---|---|---|---|---|---|---|
| | Stage for | Mass | Stage for | Mass | of Acetic | Duty, |
| | Highest | Ratio of | Lowest | Ratio of | Acid to | kJ/kg |
| | Acetic Acid | GDA:HEA at | Acetic Acid | GDA:HEA at | Highest | GDA in |
| Run | Feed | Feed Stage | Feed | Feed Stage | Feed, % | Product |
| 1* | 15 | 0.46 | 20 | 3.62 | 50 | 610 |
| 2* | 17 | 1.26 | 20 | 4.61 | 50 | 610 |
| 3* | 20 | 2.61 | 23 | 9.34 | 50 | 616 |
| 4 | 20 | 1.21 | 27 | 26.46 | 50 | 604 |
| 5 | 20 | 0.64 | 29 | 43.38 | 50 | 600 |
| 6 | 20 | 0.71 | 30 | 93.97 | 50 | 596 |
| 7 | 17 | 0.76 | 29 | 43.34 | 50 | 594 |
| 8 | 15 | 0.49 | 29 | 60.85 | 50 | 601 |
| 9 | 17 | 0.26 | 29 | 60.02 | 30 | 588 |
| 10 | 17 | 0.19 | 29 | 59.75 | 20 | 594 |
| 11 | 17 | 0.39 | 29 | 60.49 | 40 | 591 |
| 12 | 17 | 0.71 | 29 | 61.38 | 60 | 599 |
| 13* | | | 17 | 2.09 | 100 | 621 |
| 14* | | | 20 | 3.9 | 100 | 623 |
| 15* | | | 23 | 8.06 | 100 | 640 |
| 16* | | | 27 | 23.72 | 100 | 673 |

*Runs 1 through 3 and 13 through 16 are comparative runs

The results of the simulation using one or two feed points are provided in Table 2.

TABLE 2

| | Distillation | Distillation | Distillation | | Reboiler |
|---|---|---|---|---|---|
| | Stage for | Stage for | Stage for | | Duty, |
| | Highest | Transition | Lowest | Ratio of Acetic Acid of | kJ/kg |
| | Acetic Acid | Acetic Acid | Acetic Acid | Highest:Middle:Lowest | GDA in |
| Run | Feed | Feed | Feed | Feed Stage | Product |
| 17 | 20 | 26 | 29 | 0.84109 | 595 |
| 18 | 17 | 26 | 29 | 25:5:70 | 587 |
| 19 | 14 | 26 | 29 | 15:30:55 | 592 |
| 20 | 20 | 23 | 29 | 25:5:70 | 592 |
| 21 | 17 | 23 | 29 | 25:5:70 | 587 |
| 22 | 14 | 23 | 29 | 0.63281 | 591 |
| 23 | 17 | 20 | 29 | 0.83767 | 587 |
| 24 | 14 | 20 | 29 | 0.42795 | 590 |
| 25 | 14 | 17 | 29 | 0.22309 | 588 |
| 26 | 20 | 23 | 26 | 30:5:65 | 604 |
| 27 | 17 | 23 | 26 | 25:5:70 | 589 |
| 28 | 14 | 23 | 26 | 0.6294 | 592 |
| 29 | 17 | 20 | 26 | 0.83767 | 589 |
| 30 | 14 | 20 | 26 | 0.6294 | 592 |
| 31 | 14 | 17 | 26 | 0.22309 | 590 |
| 32 | 17 | 20 | 23 | 25:5:70 | 596 |
| 33 | 14 | 20 | 23 | 0.6294 | 594 |
| 34 | 14 | 17 | 23 | 0.42795 | 593 |
| 35 | 14 | 17 | 20 | 0.6294 | 599 |

TABLE 3

| | | | Lower section | Lower section |
|---|---|---|---|---|
| | | Middle | feed at next to last | feed at fourth to |
| | | section | stage, Reboiler | last stage Reboiler |
| | No. of | feed | Duty, kJ/kg GDA | Duty, kJ/kg GDA |
| Run | Stages | stage | in Product | in Product |
| 36 | 30 | 17 | 610 | 608 |
| 37 | 22 | 15 | 622 | 635 |

As can be seen from the simulations reported in Tables 1 and 2, appreciable reductions in reboiler heat duty can be achieved by adoption of the processes of this disclosure. It is particularly notable that the most significant improvements provided by this disclosure are those where a large fraction of the acetic acid is fed to the bottom of the lower section of the reactive distillation zone. Comparative runs 13 through 16 indicate that introducing the acetic acid feed lower in the reactive distillation zone increases reboiler heat duty. Yet, when a portion of the acetic acid feed is introduced into the transition section of the reactive distillation zone, even as small as 20 percent as is shown in Run 10, a substantial reduction in heat duty is obtained. Comparing Run 16, where 100% of the acetic acid feed is to distillation stage 27, to Run 10, an energy savings of about 12 percent is realized.

The use of three, acetic acid feed points in Runs 17 to 35 exemplify that once understanding the principles of this disclosure, the operator has flexibility in introducing acetic acid into more than one tray in each of the lower section and transition section of the reactive distillation zone. Moreover, based upon the distillation stage(s) to which acetic acid is fed to the lower section of the reactive distillation zone, the operator can proportion the feed to the other stages to, for instance, optimize the savings in reboiler heat duty.

Runs 36 and 37 show the effect of adding distillation stages beyond those required to achieve 5 mass percent 2-hydroxyethyl acetate in the tails. In each, 19 percent of the acetic acid is provided to the middle section. Runs 36 and 37 illustrate the enhanced benefits provided by the use of two or more acetic acid feed points as the number of distillation stages in the reactive distillation column is reduced.

Although the disclosure has been described with reference to preferred implementations, persons skilled in the art will recognize that changes may be made in form and detail without departing from the spirit and scope of the disclosed apparatus, systems, and methods.

1. A continuous process for making ethylene glycol diacetate by esterification of monoethylene glycol to 2-hydroxyethyl acetate and the esterification of 2-hydroxyethyl acetate to ethylene glycol diacetate, comprising continuously feeding monoethylene glycol and acetic acid to a reactive distillation zone having a plurality of distillation stages, said reactive distillation zone having an upper section, an intermediate section and a lower section and being under reactive distillation conditions including the presence of esterification catalyst to provide an overhead from the upper section of the reactive distillation zone comprising water and acetic acid, which overhead is continuously withdrawn from the reactive distillation zone, and a bottoms fraction, which bottoms fraction is continuously withdrawn from the lower section and contains ethylene glycol diacetate,
wherein at least about 90 percent of the monoethylene glycol is converted to ethylene glycol diacetate, and
wherein the monoethylene glycol is fed to the upper section of the reactive distillation zone and a portion of the acetic acid is fed to the lower section of the reactive distillation zone and a portion of the acetic acid is fed to the intermediate section of the reactive distillation zone.

2. The process of claim 1 wherein at least about 40 percent of the acetic acid is fed to the lower section of the reactive distillation column.

3. The process of claim 2 wherein at least about 20 percent of the acetic acid is fed to the transition section of the reactive distillation zone.

4. The process of claim 1 wherein a portion of the acetic acid is fed to one or more distillation stages in the intermediate section of the reactive distillation zone, said stages having a liquid phase in which the mole ratio of ethylene glycol diacetate to 2-hydroxyethyl acetate between about 0.1:1 and 1.5:1.

5. A continuous process for making ethylene glycol diacetate by esterification of monoethylene glycol to 2-hydroxyethyl acetate and the esterification of 2-hydroxyethyl acetate to ethylene glycol diacetate, comprising continuously feeding monoethylene glycol and acetic acid to a reactive distillation zone having a plurality of distillation stages, said reactive distillation zone having an upper section having a plurality of distillation stages including a top-most distillation stage, an intermediate section having a plurality of distillation stages and a lower section having a plurality of distillation stages including 3 distillation stages at its bottom portion, and being under reactive distillation conditions including the presence of esterification catalyst to provide an overhead from the upper section of the reactive distillation zone comprising water and acetic acid, which overhead is continuously withdrawn from the reactive distillation zone, and a bottoms fraction, which bottoms fraction is continuously withdrawn from the lower section, containing ethylene glycol diacetate,
wherein at least about 90 percent of the monoethylene glycol is converted to ethylene glycol diacetate, and
wherein (i) at least a portion of the monoethylene glycol is fed to the top most distillation stage the upper section of the reactive distillation zone, (ii) between about 40 and 80 percent of the acetic acid is fed to the lower section of the reactive distillation zone and (iii) a portion of the acetic acid is fed to the intermediate section of the reactive distillation zone.

6. The process of claim 5 wherein at least about 75 percent of the monoethylene glycol is fed to the top-most distillation stage.

7. The process of claim 5 wherein the one or more distillation stages in the intermediate section of the reactive distillation zone to which a portion of the acetic acid is fed have a liquid phase in which the mole ratio of ethylene glycol diacetate to 2-hydroxyethyl acetate between about 0.1:1 and 1.5:1.

8. The process of claim 5 wherein at least about 40 percent of the acetic acid is fed to at least one of the 3 distillation stages in the lower section of the reactive distillation zone.

9. A continuous process for making ethylene glycol diacetate from monoethylene glycol and acetic acid by reactive distillation comprising:
a. continuously supplying to a reactive distillation zone acetic acid and at least one of monoethylene glycol and 2-hydroxyethyl acetate, said reactive distillation zone providing contact between acetic acid and at least one of monoethylene glycol and 2-hydroxyethyl acetate under esterification conditions including the presence of esterification catalyst to provide an overhead containing water and acetic acid and to provide an ethylene glycol diacetate product;
b. continuously withdrawing the overhead containing water from the reactive distillation zone; and
c. continuously withdrawing from the reactive distillation zone ethylene glycol diacetate product,
wherein the overhead from the reactive distillation zone is subjected to fractional distillation in a non-reactive rectification zone having a substantial absence of esterification catalyst, to separate water as an overhead and acetic acid as a liquid phase passing to the reactive distillation zone, and between about 10 to 50 percent of the monoethylene glycol supplied to the reactive distillation zone is passed to the non-reactive rectification zone and becomes a portion of the liquid phase passing to the reactive distillation zone.
